(19) United States
(12) Patent Application Publication
(10) Pub. No.: US 2005/0009064 A1
(43) Pub. Date: Jan. 13, 2005

(54) Nucleic acid detection and quantification using terminal transferase based assays

(76) Inventors: Arjang Hassibi, Palo Alto, CA (US); Siavash Ghazvini, Menlo Park, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/844,674

(22) Filed: May 13, 2004

Related U.S. Application Data
(60) Provisional application No. 60/470,347, filed on May 13, 2003.

(51) Int. Cl. C12Q 1/68; C12P 19/34

(52) U.S. Cl. 435/6; 435/91.2

(57) ABSTRACT

The present invention concerns methods of detecting, identifying and/or quantifying nucleic acids using a terminal transferase based assay. Terminal transferase adds nucleotides to the 3′ end of single-stranded DNA or the 3′ overhang of restricted double-stranded DNA, resulting in production of one molecule of pyrophosphate for each nucleotide incorporated. In various embodiments, a bioluminescence regenerative cycle (BRC) may be used to measure the amount of pyrophosphate produced by terminal transferase activity. In BRC, steady state levels of bioluminescence result from processes that produce pyrophosphate. Pyrophosphate reacts with APS in the presence of ATP sulfurylase to produce ATP. The ATP reacts with luciferin in a luciferase-catalyzed reaction, producing light and regenerating pyrophosphate. The pyrophosphate is recycled to produce ATP and the regenerative cycle continues. During the course of the cycle a steady state is achieved wherein concentrations of ATP and pyrophosphate and the rate of light production remain relatively constant. In preferred embodiments, photon emission is integrated over a time interval to determine the number of target molecules present in the initial sample. In certain embodiments, the targets to be detected may comprise reporter oligonucleotides attached to biomolecules, such as proteins, peptides, antibodies, ligands, etc. In other embodiments, one or more of the enzymes used may be thermostable enzymes.

This is a non provisional application based on the provisional application Ser. No. 60/470,347 filed on May 13, 2003 and claims priority thereof.

### BACKGROUND OF THE INVENTION

1. Field of the Invention

The present invention relates to the field of nucleic acid detection and/or quantification. More particularly, the present invention concerns novel approaches to detection and/or quantification of nucleic acids using terminal transferase based assays. The nucleic acids to be quantified may be attached to target proteins or other biomolecules of interest.

2. Description of Related Art

Methods of precise and highly sensitive detection and/or quantification of nucleic acids are of use for a variety of medical, forensic, epidemiological, public health, biological warfare and other applications. A variety of molecular biology and genomic techniques would benefit from the availability of precise and sensitive methods for nucleic acid detection and/or quantification.

DNA microarrays provide a platform for detecting and identifying nucleic acids by hybridization with sequence specific oligonucleotide probes attached to chips in precise arrays. (E.g., Schena et al., Science 270:467-470, 1995; Shalon et al., Genome Res. 6:639-645, 1996; Pease et al., Proc. Natl. Acad. Sci. USA 91:5022-26, 1994). Microarray technology is an extension of previous hybridization-based methods, such as Southern and Northern blotting, that have been used to identify and quantify nucleic acids in biological samples (Southern, J. Mol. Biol. 98:503-17, 1975; Pease et al., Proc. Natl. Acad. Sci. USA 93:10614-19, 1996). Identification of a target nucleic acid in a sample typically involves fluorescent detection of the nucleic acid hybridized to an oligonucleotide at a particular location on the array. Fluorescent detection is too insensitive to detect very low levels of a target nucleic acid in a sample. It is also more qualitative than quantitative. More accurate and sensitive methods for nucleic acid quantification are needed.

Real time PCR™ (polymerase chain reaction) is another technique for which accurate and sensitive detection and/or quantification are needed (e.g., Model 770 TaqMan® system, Applied Biosystems, Foster City, Calif.). Typically, if the target of interest is present, it will be amplified by replication using flanking primers and a nucleic acid polymerase. A probe, which may consist of a complementary oligonucleotide with attached reporter and quencher dyes, is designed to bind to the amplified target nucleic acid between the two primer-binding sites. The nuclease activity of the polymerase cleaves the probe, resulting in an increase in fluorescence of the reporter dye after it is separated from the quencher. PCR based fluorescence detection of target nucleic acids is more sensitive, due to the amplification effect of the technique. However, detection and/or quantification of the target may be complicated by a variety of factors, such as contaminating nuclease activity or variability in the efficiency of amplification.

Single nucleotide polymorphisms (SNPs) are of increasing interest in molecular biology, genomics and disease diagnostics. SNP detection may be used for haplotype construction in genetic studies to identify and/or detect genes associated with various disease states, as well as drug sensitivity or resistance. SNPs may be detected by a variety of techniques, such as DNA sequencing, fluorescence detection, mass spectrometry or DNA microarray hybridization (e.g., U.S. Pat. Nos. 5,885,775; 6,368,799). Existing methods of SNP detection may suffer from insufficient sensitivity or an unacceptably high level of false positive and/or false negative results. A need exists for more sensitive and accurate methods of detecting SNPs.

Pyrophosphate based detection systems have been used for DNA sequencing (e.g., Nyren and Lundin, Anal. Biochem. 151:504-509, 1985; U.S. Pat. Nos. 4,971,903; 6,210,891; 6,258,568; 6,274,320, each incorporated herein by reference). The method uses a coupled reaction wherein pyrophosphate is generated by an enzyme-catalyzed process, such as nucleic acid polymerization. The pyrophosphate is used to produce ATP, in an ATP sulfurylase catalyzed reaction with adenosine 5′-phosphosulphate (APS). The ATP in turn is used for the production of light in a luciferin-luciferase coupled reaction. However, the “pyrosequencing” technique is based on sequential addition of single nucleotides, in the presence of nucleotide degrading enzymes to remove any unincorporated nucleotides (U.S. Pat. Nos. 6,210,891 and 6,258,568). This results in low levels of light emission, with relatively low sensitivity, and requires a complex and expensive apparatus to perform the assay.

Certain embodiments of the present invention involve quantification of target proteins, peptides or other biomolecules that are tagged with reporter oligonucleotides. A number of methods are known for protein identification, detection and quantification, such as SDS-polyacrylamide gel electrophoresis, capillary electrophoresis, limited proteolysis and tandem array mass spectrometry, enzyme assay, cell-based assays and a wide of immunologic techniques such as Western blotting and ELISA. In certain instances, such techniques may require partial or even full purification of the protein of interest before it can be quantified. In other cases, the detection methods, such as immunoassay, may show cross-reactivity with other proteins that may be present in a complex mixture. Immunoassays also require that one or more antibodies be prepared against the target protein of interest, a laborious and time-consuming process. Improved methods for detection, identification and/or quantification of biomolecules, such as nucleic acids or oligonucleotide-tagged proteins, peptides, etc. are needed. Preferably such methods would be simple, inexpensive and rapid, with high sensitivity and specificity for the target molecule to be detected.

### SUMMARY OF THE INVENTION

The present invention fulfills an unresolved need in the art by providing methods for accurately detecting, identifying and/or quantifying nucleic acid sequences, using terminal transferase based assays. The disclosed methods provide increased sensitivity and accuracy of target molecule detection, identification and/or quantification compared to prior art methods.

In certain embodiments of the invention, the methods may comprise obtaining at least one sample suspected of containing one or more target nucleic acids. The target nucleic acid(s) may be captured and/or isolated by a variety of known techniques, such as sequence specific hybridization of target nucleic acids with one or more capture probes. In alternative embodiments of the invention, the target nucleic acid may comprise an oligonucleotide tag attached to another biomolecule, such as a protein, peptide, antibody, antigen, enzyme, binding protein, ligand, substrate and/or inhibitor. The target nucleic acid may be captured and/or isolated using known techniques, such as antibody-antigen binding, protein-ligand binding, enzyme-inhibitor or enzyme-substrate binding, etc.

In other alternative embodiments of the invention, target proteins or other biomolecules may be detected by binding to an aptamer. Aptamers are oligonucleotides that exhibit specific binding interactions not based on standard Watson-Crick basepair formation and are therefore similar to antibodies in their binding characteristics. Aptamers may be derived by an in vitro evolutionary process called SELEX (e.g., Brody and Gold, Molecular Biotechnology 74:5-13, 2000). Aptamers may be produced by known methods (e.g., U.S. Pat. Nos. U.S. Pat. Nos. 5,270,163; 5,567,588; 5,670,637; 5,696,249; 5,843,653) or obtained from commercial sources (e.g, Somalogic, Boulder, Colo.). Aptamers are relatively small molecules on the order of 7 to 50 kDa. Because they are small, stable and not as easily damaged as proteins, they may be well suited for assays involving binding to the surface of a solid matrix. Because aptamers may be comprised of DNA, they can serve as substrates for terminal transferase activity and chemiluminescent detection as disclosed herein.

The captured and/or isolated target nucleic acid may be detected, identified and/or quantified using a variety of terminal transferase based assay methods. In preferred embodiments of the invention, the target nucleic acid may be detected, identified and/or quantified using a bioluminescence regenerative cycle (BRC) assay, discussed in more detail below. Terminal transferase may be added to the target nucleic acid in the presence of nucleotides (dNTPs). Terminal transferase will add nucleotides to the 3′ end of single-stranded DNA (ssDNA) or the 3′ overhangs of double-stranded DNA that has been treated, for example, with a restriction endonuclease. Terminal transferase may also add nucleotides to blunt-ended double-stranded DNA or the recessed 3′ ends of restricted double-stranded DNA, with lower efficiency. Incorporation of nucleotides by terminal transferase results in generation of pyrophosphate (PPi), with one molecule of PPi generated for each nucleotide incorporated.

In more preferred embodiments, the pyrophosphate producing reaction is allowed to proceed to completion before BRC analysis. Once the reaction is complete, the pyrophosphate is reacted with APS (adenosine 5′-phosphosulfate) in the presence of ATP sulfurylase to produce ATP and sulphate. The ATP is reacted with oxygen and luciferin in the presence of luciferase to yield oxyluciferin, AMP and pyrophosphate. The PPi may react again with APS to regenerate ATP. For each molecule of pyrophosphate that is cycled through BRC, a photon of light is emitted and one molecule of pyrophosphate is regenerated. Because of the relative kinetic rates of luciferase and ATP sulfurylase, a steady state is reached in which the concentrations of ATP and pyrophosphate and the level of photon output remain relatively constant over an extended period of time. The number of photons may be counted (integrated) over a time interval to determine the number of target nucleic acids in the sample. The very high sensitivity of BRC is related in part to the integration of light output over time, in contrast to other methods that measure light output at a single time point or at a small number of fixed time points. The ability to vary the length of time over which photon integration occurs also contributes to the very high dynamic range for nucleic acid molecule quantification. The detection noise is also significantly reduced by increasing the length of integration.

In other preferred embodiments of the invention, the steady state light output is subjected to data analysis involving integration of light output over a time interval, providing an accurate and highly sensitive method of quantifying the number of target nucleic acids (3′ termini) in the sample. In various embodiments of the invention, light output by BRC may be corrected for background light emission (for example, by PPi contaminating one or more reagents) by comparing terminal transferase mediated photon emission with the background photon emission.

In certain embodiments of the invention, thermostable enzymes may be used in a BRC detection method. Thermostable forms of terminal transferase, ATP sulfurylase and luciferase are disclosed herein and may be used for either isothermal processes or thermal cycling reactions.

The invention is not limited to BRC assay of terminal transferase activity. It will be apparent to the skilled artisan that many different methods of assaying terminal activity are known and may be used in the practice of the disclosed methods, such as incorporation of fluorescently tagged nucleotides and fluorescence spectroscopy; incorporation of radioactively tagged nucleotides and liquid scintillation counting or other assay; incorporation of Raman labels and Raman spectroscopy; incorporation of NMR labels and nuclear magnetic resonance assay, and many other techniques known in the art. In various embodiments of the invention, multi-color detection methods may be employed, using nucleotides tagged with different color fluorophores.

In some embodiments of the invention, the disclosed methods are of use for a wide variety of applications for which nucleic acid detection, identification and/or quantification is desired. Such applications include, but are not limited to, measuring gene expression levels, detecting and/or quantifying pathogens in a sample, performing real-time PCR™ analysis and detecting single nucleotide polymorphisms (SNPs).

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 illustrates the capture and detection of DNA target (1a-3a) or antibody linked with a DNA molecule (1b-3b) using a solid surface, followed by extension of 3′ terminus by terminal transferase.

FIG. 2. illustrates a nucleic acid detection procedure using terminal transferase and BRC. The terminal transferase step is terminated and PPi is determined by BRC assay.

FIG. 3. illustrates a nucleic acid detection procedure using real-time BRC assay and terminal transferase.

### DESCRIPTION OF ILLUSTRATIVE EMBODIMENTS

Definitions

Terms that are not otherwise defined herein are used in accordance with their plain and ordinary meaning.

As used herein, “a” or “an” may mean one or more than one of an item.

As used herein, “luminescence” refers to the emission of light that does not derive energy from the temperature of the emitting body (i.e., emission of light other than incandescent light). “Luminescence” includes, but is not limited to, fluorescence, phosphorescence, thermoluminescence, chemiluminescence, electroluminescence and bioluminescence. “Luminescent” refers to an object that exhibits luminescence. In preferred embodiments, the light is in the visible spectrum. However, the present invention is not limited to visible light, but includes electromagnetic radiation of any frequency.

“Nucleic acid” means either DNA, RNA, single-stranded, double-stranded or triple stranded and any chemical modifications thereof. Virtually any modification of the nucleic acid is contemplated by this invention. “Nucleic acid” encompasses, but is not limited to, oligonucleotides and polynucleotides. Within the practice of the present invention, a “nucleic acid” may be of any length.

Terminal Transferase Based Assays

Described herein are enzymatic methods to detect and/or quantify the presence of DNA molecules and/or other biological species linked to DNA molecules in a biological sample, by means of terminal transferase enzyme. Sources of and general methods applicable to terminal transferase assays are known in the art (e.g., Chang and Bollum, CRC Crit. Rev. Biochem., 21, 27-52, 1986; Roychoudhury et al., Nucl. Acids Res. 3, 101-116, 1976; Tu and Cohen, Gene 10, 177-183, 1980; Boule et al., J. Biol. Chem. 276, 31388-31393, 2001).

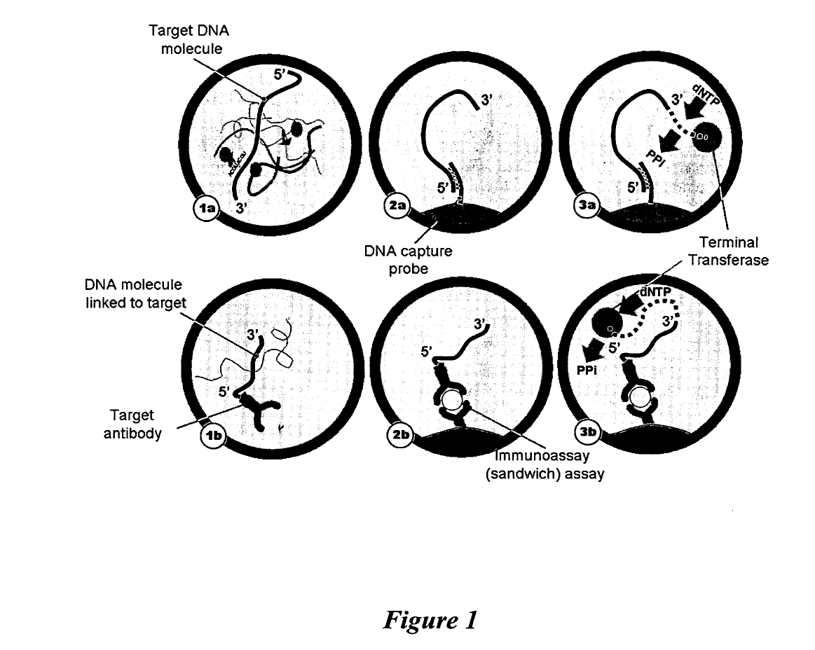

The general approach is to first capture or isolate one or more specific DNA target molecules, or a target moiety containing DNA probes (e.g., antibody molecules linked with a DNA oligonucleotide) from the biological sample. The isolation can be carried out by various solid surface methods (e.g. capturing probe-coated magnetic beads), affinity matrixes, or electrophoretic processes. Once a target DNA has been captured or isolated terminal transferase enzyme is added in the presence of nucleotides (dNTPs). Terminal transferase catalyzes the addition of dNTPs to the 3′ terminus of DNA. This enzyme works on single-stranded DNA (ssDNA), including 3′ overhangs of double-stranded DNA (dsDNA). Its activity therefore resembles a DNA polymerase which does not require a primer, avoiding the need for a separate primer hybridization procedure. Because the enzyme can be used with double-stranded DNA, it does not require the separate isolation of single-stranded DNA. A general scheme for methods of use of terminal transferase for nucleic acid detection and/or quantitation is illustrated in FIG. 1.

As disclosed in FIG. 1, the target nucleic acid can either be free (1a-3a) or can be attached to another molecule, such as an antibody (1b-3b). In cases where the target is an RNA molecule, such as a messenger RNA (mRNA), the RNA may be converted to cDNA using reverse transcriptase, according to known protocols (e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). The target nucleic acid may be captured, for example, by hybridization to a sequence specific capture probe (2a). Alternatively, target nucleic acids attached to another molecule may be captured by a variety of known immobilization methods, such as sandwich immunoassay (2b). Once captured, the substrate may be washed to remove unbound nucleic acids and the bound target may be extended using terminal transferase (3a, 3b). Where capture oligonucleotides are used, the 3′ end may be blocked, for example using dideoxy nucleotides, to prevent the terminal transferase from extending unhybridized capture probes.

The rate of terminal transferase mediated dNTP incorporation into the captured strand depends on the concentration of the enzyme, nucleotides and the relative amount of captured 3′ termini (which is in turn a function of the amount of target nucleic acid in the sample). Given the accurate determination of terminal transferase activity in a fixed time interval, and the initial nucleotide and enzyme concentrations, it is possible to correlate the measured terminal transferase activity with the concentration of target nucleic acid (total amount of 3′ terminus) in the sample.

Terminal transferase based assays measure the number of 3′ termini of DNA molecules in the sample, independent of the DNA being the actual target or just a reporter species linked to a secondary target. The enzyme can in theory incorporate unlimited number of nucleotides into the strand. However in a fixed time interval, depending on the activity of the enzyme, this number will be within a given deterministic range. A typical terminal transferase reaction may be performed, for example, at 20° C. in buffer containing 20 mM Tris acetate (pH 7.9) and 50 mM potassium acetate, supplemented with 1.5 mM CoCl₂ . Alternative assay conditions include 50 mM potassium acetate, 20 mM Tris-acetate (pH 7.9), 10 mM magnesium acetate and 1 mM dithiothreitol, incubated at 37° C. Additional conditions suitable for assay of terminal transferase activity are known (see, e.g., Chang and Bollum, 1986; Roychoudhury et al., 1976; Tu and Cohen, 1980; Boule et al., 2001).

Although a preferred substrate for terminal transferase is protruding 3′ ends, it will also less efficiently add nucleotides to blunt and 3′-recessed ends of ssDNA or dsDNA fragments. Cobalt is the necessary cofactor for activity of this enzyme. Terminal transferase may be purchased commercially (e.g., Fermentas, Inc., Hanover, Md.; Promega, Madison, Wis.; Stratagene, La Jolla, Calif.) and is usually produced by expression of the bovine gene in E. coli.

The growth of a DNA strand in a terminal transferase based assay can potentially result in a variety of detectable phenomena. Exemplary measurable changes produced by enzyme activity include, but are not limited to, intrinsic characteristics of the growing molecule itself (e.g., molecular mass, overall charge) as well as natural products of the incorporation reaction (e.g. PPi). Alternatively other effects can be measured using extrinsic modifications. These may include various labels or fluorogenic species attached to or incorporated into the nucleotide substrates. In preferred embodiments, the BRC assay system is used to detect PPi generated by terminal transferase activity.

BRC Detection Method

The BRC method involves the luminescent detection of pyrophosphate (PPi) molecules released from an enzyme-catalyzed reaction, such as terminal transferase activity. As part of the technique, a bioluminescence regenerative cycle (BRC) is triggered by the release of inorganic pyrophosphate (PPi). Further details on the BRC method are disclosed in U.S. patent application Ser. No. 10/186,455, filed Jun. 28, 2002, the entire text of which is incorporated herein by reference.

The BRC regenerative cycle can be utilized with any reaction that generates pyrophosphate (PPi). The PPi generated reacts with APS, catalyzed by ATP-sulfurylase enzyme, which results in the production of ATP and inorganic sulphate. In another reaction, luciferin and luciferase consume ATP as an energy source to generate light, AMP and oxyluciferin and to regenerate PPi. Thus, after each BRC cycle, a quantum of light is generated for each molecule of PPi in solution, while the net concentration of ATP in solution remains relatively stable and is proportional to the initial concentration of PPi. In the course of the reactions, APS and luciferin are consumed and AMP and oxyluciferin are generated, while ATP sulfurylase and luciferase remain constant. The invention is not limited as to the type of luciferase used. Although certain disclosed embodiments utilized firefly luciferase, any luciferase known in the art may be used in the disclosed methods.

As a result of the BRC process, the photon emission rate remains steady and is a monotonic function of the amount of PPi in the initial mixture. For very low concentrations of PPi (10⁻⁸ M or less), the total number of photons generated in a fixed time interval is proportional to the number of PPi molecules. Where PPi is generated by the activity of terminal transferase on a target nucleic acid, the number of photons generated in a fixed time interval is proportional to the quantity of the target nucleic acid present in the sample.

The basic concept of enzymatic light generation from PPi molecules was introduced almost two decades ago (Nyren and Lundin, 1985; Nyren, Anal. Biochem. 167:235-238, 1987). Pyrophosphate based luminescence has been used for DNA sequencing (Ronaghi et al., Anal. Biochem. 242:84-89, 1996) and SNP detection (Nyren et al., Anal. Biochem. 244:367-373, 1997). The present methods provide additional procedures for accurately detecting, identifying and/or quantifying specific target nucleic acids, in the presence of contaminants and detector noise. The system and methods have an intrinsic controllable dynamic range up to seven orders of magnitude and are sensitive enough to detect target nucleic acids at attomole (10⁻¹⁸) or lower levels. Because the BRC process allows integration of steady state photon emission over time, the sensitivity of target nucleic acid detection is many orders of magnitude higher than pyrosequencing and other techniques, which utilize detection at single time points and may use apyrase and/or single nucleotide addition to generate short pulses of light emission, in contrast to a sustained emission of light that is monotonically dependent on the starting concentration of PPi.

Enzymatic Bioluminescence Cycle

To generate photons from pyrophosphate, ATP-sulfurylase (Ronesto et al., Arch. Biochem. Biophys. 290:66-78, 1994; Beynon et al. Biochemistry, 40, 14509-14517, 2001) is used to catalyze the transfer of the adenylyl group from APS to PPi, producing ATP and inorganic sulfate.

Next, luciferase catalyzes the slow consumption of ATP, luciferin and oxygen to generate a single photon (λₘₐₓ=562 nm, Q.E.≈0.88) per ATP molecule, regenerating a molecule of PPi and producing AMP, CO₂ and oxyluciferin (Brovko et al., Biochem. (Moscow) 59:195-201, 1994). Because the luciferase reaction is significantly slower than the ATP-sulfurylase reaction, in the presence of sufficient amounts of the substrates APS and luciferin a steady state cycle should be maintained, in which the concentration of ATP and the resulting levels of light emission remain relatively constant for a considerable time.

Because the steady-state photon emission is proportional to the initial concentration of PPi, the presence of minute amounts of PPi produced by a terminal transferase reaction should result in a detectable shift in baseline luminescence, even in the presence of considerable amounts of noise. The number of photons generated over time by the BRC cycle can potentially be orders of magnitude higher than the initial number of PPi molecules, which makes the system extremely sensitive compared to prior art methods of nucleic acid quantification. The increased sensitivity is provided by having a time-dependent amplification of light emission for each molecule of PPi present at the start of the BRC cycle.

As applied to terminal transferase mediated assays, the number of PPi molecules released in a sample is equal to the number of nucleotides incorporated onto the 3′ terminus of the target or reporter DNA. In alternative embodiments of the invention, either the terminal transferase process may be terminated after a specified time interval, or alternatively the rate of PPi generation may be measured in real time.

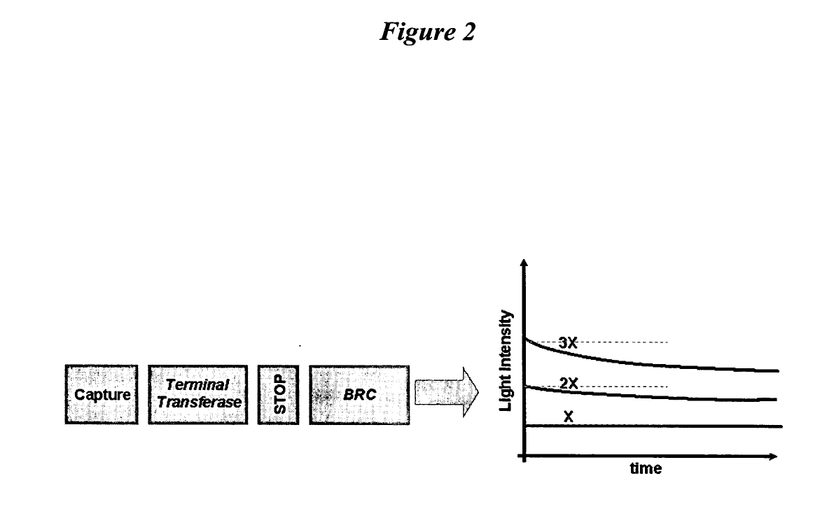

In the first approach, where terminal transferase activity is terminated (for example by raising the temperature above 70° C.) the intensity of light emission from BRC will be stable, with a steady-state level that is proportional to target concentration (FIG. 2). This is because of the finite amount of PPi generated by terminal transferase and the inherent characteristics of the BRC assay.

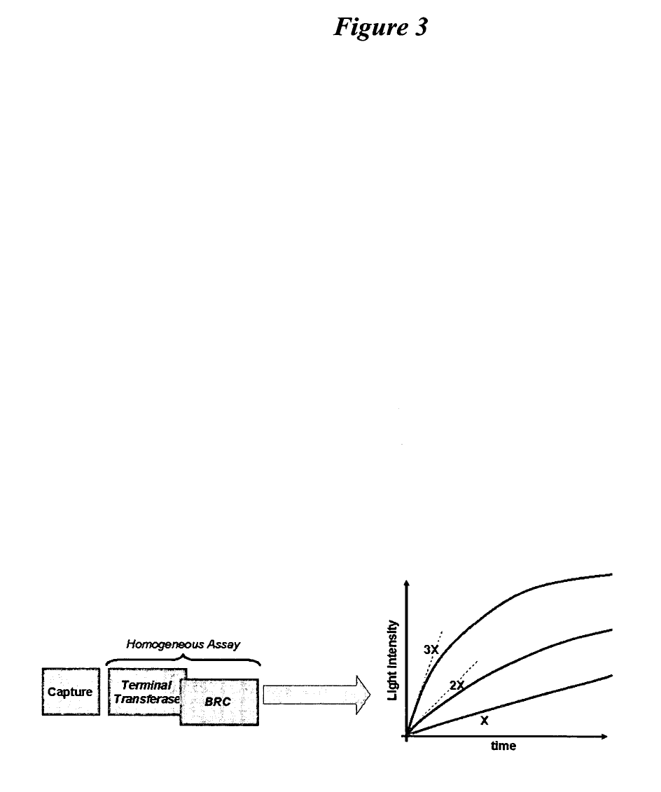

In an alternative embodiment, the BRC assay and terminal tranferase activity are conducted simultaneously in one homogeneous assay. As shown in FIG. 3, the intensity of emitted light will increase as terminal tranferase adds more dNTPs to the target nucleic acid, resulting in an increased PPi concentration with time. In this approach the kinetics of the terminal transferase reaction, as measured by BRC light emission, provides a measure of target nucleic acid concentration. An advantage of this embodiment is that light emission continues to increase until the enzyme runs out of substrate or is inhibited by some other process. The simultaneous assay thus offers the advantage of increased signal strength and potentially increased sensitivity of the assay.

Thermostable Enzymes

In certain embodiments of the invention, thermostable enzymes may be used for the terminal transferase and/or BRC processes. A thermostable terminal transferase activity is exhibited, for example, by most thermostable Taq polymerase enzymes. Thermostable Taq polymerase may be commercially obtained, for example from Promega (Madison, Wis.), Gentaur (Brussels, Belgium) or Roche Applied Science (Indianapolis, Ind.). The terminal transferase activity of Taq polymerase shows a preference for adding adenines to the 3′ end of DNA, including blunt ended double-stranded DNA.

Amino acid and DNA sequence data (GenBank Accession NO. AAC07134) for a thermostable form of ATP sulfurylase have been reported (Hanna et al., Arch. Biochem. Biophys. 406:275-288, 2002). Hanna et al. (2002) also report methods for cloning and purification of a thermostable ATP sulfurylase from the hyperthermophilic chemolithotroph Aquifex aeolicus. The enzyme is reported to be highly heat stable, with a half life of more than one hour at 90° C.

Thermostable luciferase may be obtained from commercial sources (Promega; ultraglow recombinant luciferase, catalog No. E140X). The luciferase has been observed to be stable to temperatures as high as 95° C.

In various embodiments of the invention, a thermostable terminal transferase may be used with detection methods that do not involve BRC, such as incorporation of fluorescently tagged nucleotides into a target oligonucleotide and/or nucleic acid sequence. In alternative embodiments, thermostable ATP sulfurylase and luciferase may be used for BRC detection of PPi generated by any enzymatic process. As discussed in U.S. Ser. No. 10/186,455, incorporated herein by reference, pyrophosphate may be generated by a variety of processes, such as reverse transcriptase activity, polymerase chain reaction, DNA polymerase mediated DNA replication, and/or by terminal transferase activity.

Nucleic Acids

Samples comprising target nucleic acids may be prepared by any technique known in the art. In certain embodiments, the analysis may be performed on crude sample extracts, containing complex mixtures of nucleic acids, proteins, lipids, polysaccharides and other compounds. Such samples are likely to contain contaminants that could potentially interfere with the BRC process. In preferred embodiments, the target nucleic acids may be partially or fully separated from other sample constituents before initiating the BRC analysis.

Methods for partially or fully purifying nucleic acids from complex mixtures, such as cell homogenates or extracts, are well known in the art. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). Generally, cells, tissues or other source material containing nucleic acids are first homogenized, for example by freezing in liquid nitrogen followed by grinding in a mortar and pestle. Certain tissues may be homogenized using a Waring blender, Virtis homogenizer, Dounce homogenizer or other homogenizer. Crude homogenates may be extracted with detergents, such as sodium dodecyl sulphate (SDS), Triton X-100, CHAPS (3-[(3-cholamidopropyl)-dimethylammonio]-1-propane sulfonate), octylglucoside or other detergents known in the art. As is well known, nuclease inhibitors such as RNase or DNase inhibitors may be added to prevent degradation of target nucleic acids.

Extraction may also be performed with chaotrophic agents such as guanidinium isothiocyanate, or organic solvents such as phenol. In some embodiments, protease treatment, for example with proteinase K, may be used to degrade cell proteins. Particulate contaminants may be removed by centrifugation or ultracentrifugation. Dialysis against aqueous buffer of low ionic strength may be of use to remove salts or other soluble contaminants. Nucleic acids may be precipitated by addition of ethanol at −20° C., or by addition of sodium acetate (pH 6.5, about 0.3 M) and 0.8 volumes of 2-propanol. Nucleic acids may be collected by centrifugation or other known methods, such as preparative agarose gel electrophoresis or use of a commercially available column for preparation of plasmids or other vectors. The skilled artisan will realize that the procedures listed above are exemplary only and that many variations may be used, depending on the particular type of nucleic acid to be analyzed.

In certain embodiments, the target nucleic acids of interest may be immobilized on a substrate as discussed above. Nucleic acids may be immobilized, for example, by hybridization with a capture oligonucleotide probe.

Pathogen Detection

In certain embodiments of the invention, the target nucleic acid to be detected may be of a sequence specific to a pathogenic organism. Detection of a pathogen specific target nucleic acid sequence may be of use, for example, to detect and/or differentially diagnose a pathogen infection in a human or animal subject. The methods are not limited to detection of any specific pathogenic organism, and any pathogen known in the art may be detected and/or diagnosed using the disclosed methods. Exemplary pathogens within the scope of the present invention are listed in Table 1 below.

TABLE 1
Non-limiting Exemplary Pathogens

| | |
|---|---|
| Actinobacillus spp. | Burkholderia mallei |
| Actinomyces spp. | Burkholderia pseudomallei |
| Adenovirus (types 1, 2, 3, 4, 5 et 7) | Campylobacter fetus subsp. fetus |
| Adenovirus (types 40 and 41) | Campylobacter jejuni |
| Aerococcus spp. | C. coli |
| Aeromonas hydrophila | C. fetus subsp. jejuni |
| Ancylostoma duodenale | Candida albicans |
| Angiostrongylus cantonensis | Capnocytophaga spp. |
| Ascaris lumbricoides | Chlamydia psittaci |
| Ascaris spp. | Chlamydia trachomatis |
| Aspergillus spp. | Citrobacter spp. |
| Bacillus anthracis | Clonorchis sinensis |
| Bacillus cereus | Clostridium botulinum |
| Bacteroides spp. | Clostridium difficile |
| Balantidium coli | Clostridium perfringens |
| Bartonella bacilliformis | Clostridium tetani |
| Blastomyces dermatitidis | Clostridium spp. |
| Bluetongue virus | Coccidioides immitis |
| Bordetella bronchiseptica | Colorado tick fever virus |
| Bordetella pertussis | Corynebacterium diphtheriae |
| Borrelia burgdorferi | Coxiella burnetii |
| Branhamella catarrhalis | Coxsackievirus |
| Brucella spp. | Creutzfeldt-Jakob agent, Kuru |
| | agent |
| B. abortus | Crimean-Congo hemorrhagic fever |
| | virus |
| B. canis, | Cryptococcus neoformans |
| B. melitensis | Cryptosporidium parvum |
| B. suis | Cytomegalovirus |
| Brugia spp. | Dengue virus (1, 2, 3, 4) |
| Diphtheroids | Hepatitis E virus |
| Eastern (Western) equine encephalitis | Herpes simplex virus |
| virus | Herpesvirus simiae |
| Ebola virus | Histoplasma capsulatum |
| Echinococcus granulosus | Human coronavirus |
| Echinococcus multilocularis | Human immunodeficiency virus |
| Echovirus | Human papillomavirus |
| Edwardsiella tarda | Human rotavirus |
| Entamoeba histolytica | Human T-lymphotrophic virus |
| Enterobacter spp. | Influenza virus |
| Enterovirus 70 | Junin virus/Machupo virus |
| Epidermophyton floccosum, | Klebsiella spp. |
| Microsporum spp. Trichophyton spp. | Kyasanur Forest disease virus |
| Epstein-Barr virus | Lactobacillus spp. |
| Escherichia coli, enterohemorrhagic | Legionella pneumophila |
| Escherichia coli, enteroinvasive | Leishmania spp. |
| Escherichia coli, enteropathogenic | Leptospira interrogans |
| Escherichia coli, enterotoxigenic | Listeria monocytogenes |
| Fasciola hepatica | Lymphocytic choriomeningitis |
| | virus |
| Francisella tularensis | Marburg virus |
| Fusobacterium spp. | Measles virus |
| Gemella haemolysans | Micrococcus spp. |
| Giardia lamblia | Meraxella spp. |
| Giardia spp. | Mycobacterium spp. |
| Haemophilus ducreyi | Mycobacterium tuberculosis, |
| | M bovis |
| Haemophilus influenzae (group b) | Mycoplasma hominis, M orale, M. |
| Hantavirus | salivarium, M. fermentans |
| Hepatitis A virus | Mycoplasma pneumoniae |
| Hepatitis B virus | Naegleria fowleri |
| Hepatitis C virus | Necator americanus |
| Hepatitis D virus | Neisseria gonorrhoeae |
| Neisseria meningitidis | Sindbis virus |
| Neisseria spp. | Sporothrix schenckii |
| Nocardia spp. | St. Louis encephalitis virus |
| Norwalk virus | Murray Valley encephalitis virus |
| Omsk hemorrhagic fever virus | Staphylococcus aureus |
| Onchocerca volvulus | Streptobacillus moniliformis |
| Opisthorchis spp. | Streptococcus agalactiae |
| Parvovirus B19 | Streptococcus faecalis |
| Pasteurella spp. | Streptococcus pneumoniae |
| Peptococcus spp. | Streptococcus pyogenes |
| Peptostreptococcus spp. | Streptococcus salivarius |
| Plesiomonas shigelloides | Taenia saginata |
| Powassan encephalitis virus | Taenia solium |
| Proteus spp. | Toxocara canis, T. cati |
| Pseudomonas spp. | Toxoplasma gondii |
| Rabies virus | Treponema pallidum |
| Respiratory syncytial virus | Trichinella spp. |
| Rhinovirus | Trichomonas vaginalis |
| Rickettsia akari | Trichuris trichiura |
| Rickettsia prowazekii, R. canada | Trypanosoma brucei |
| Rickettsia rickettsii | Ureaplasma urealyticum |
| Ross river virus/O'Nyong-Nyong | Vaccinia virus |
| virus | |
| Rubella virus | Varicella-zoster virus |
| Salmonella choleraesuis | Venezuelan equine encephalitis |
| Salmonella paratyphi | Vesicular stomatitis virus |
| Salmonella typhi | Vibrio cholerae, serovar 01 |
| Salmonella spp. | Vibrio parahaemolyticus |
| Schistosoma spp. | Wuchereria bancrofti |
| Scrapie agent | Yellow fever virus |
| Serratia spp. | Yersinia enterocolitica |
| Shigella spp. | Yersinia pseudotuberculosis |
| Yersinia pestis | |

Enzyme Catalyzed Pyrophosphate Generation

In certain embodiments of the invention, the biomolecule dependent generation of pyrophosphate may utilize other enzymes besides terminal transferase. Within the scope of the present invention, pyrophosphate may be produced by any method known in the art. Exemplary embodiments are described below.

Primers

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

Amplification Methods

A number of template dependent processes are available to produce pyrophosphate. One of the best known methods is the polymerase chain reaction (referred to as PCR™) which is described in detail in U.S. Pat. Nos. 4,683,195, 4,683,202 and 4,800,159, and in Innis et al., 1990. Briefly, in PCR, two primer sequences are prepared which are complementary to regions on opposite complementary strands of, for example, a target nucleic acid. An excess of deoxynucleoside triphosphates are added to a reaction mixture along with a DNA polymerase, e.g., Taq polymerase. If the target sequence is present in a sample, the primers will bind to the target and the polymerase will cause the primers to be extended by adding on nucleotides. Each nucleotide incorporated results in the generation of a molecule of pyrophosphate. By raising and lowering the temperature of the reaction mixture, the extended primers will dissociate from the nucleic acid template to form amplification products, excess primers will bind to the target nucleic acid and to the amplification products and the process is repeated.

A reverse transcriptase PCR amplification procedure may be performed with mRNA as a target nucleic acid. Methods of reverse transcribing RNA into cDNA are well known and described in Sambrook et al. (1989). A molecule of pyrophosphate is generated for each nucleotide incorporated into a complementary DNA (cDNA) product. Alternative methods for reverse transcription utilize thermostable DNA polymerases.

Qbeta Replicase, described in PCT Application No. PCT/US87/00880, may also be used as another exemplary method for pyrophosphate generation. In this method, a replicative sequence of RNA that has a region complementary to that of a target nucleic acid is added to a sample in the presence of an RNA polymerase. The polymerase will copy the replicative sequence which may then be detected by pyrophosphate generation.

An isothermal method, in which restriction endonucleases and ligases are used to amplify target nucleic acid molecules that contain nucleotide 5′-[alpha-thio]-triphosphates in one strand of a restriction site may also be useful in methods of biomolecule dependent pyrophosphate generation. Walker et al., (1992).

Strand Displacement Amplification (SDA) is another method for isothermal amplification of target nucleic acids that involves multiple rounds of strand displacement and synthesis, i.e., nick translation. Other target nucleic acid amplification procedures include transcription-based amplification systems (TAS), nucleic acid sequence based amplification (NASBA) and 3SR. Kwoh et al. (1989) and PCT Application WO 88/10315.

Davey et al., European Application No. 329,822 disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H. The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman, (1990) and Ohara et al., (1989). Each of the processes discussed herein may be utilized to generate pyrophosphate, which may be assayed for example using a BRC method.

The methods disclosed herein are exemplary only. Many biomolecule dependent processes are known for pyrophosphate generation and any such known process may be used within the scope of the present invention.

Methods of Immobilization

In various embodiments, target nucleic acids and/or capture oligonucleotide probes may be attached to a solid surface (or immobilized). Immobilization of nucleic acids and/or oligonucleotides may be achieved by a variety of methods involving either non-covalent or covalent attachment. In an exemplary embodiment, immobilization may be achieved by coating a surface with streptavidin or avidin and the subsequent attachment of a biotinylated oligonucleotide or nucleic acid (Holmstrom et al., Anal. Biochem. 209:278-283, 1993). Immobilization may also occur by coating a silicon, glass or other surface with poly-L-Lys (lysine), followed by covalent attachment of either amino- or sulfhydryl-modified nucleic acids or caputure probes using bifunctional crosslinking reagents (Running et al., BioTechniques 8:276-277, 1990; Newton et al., Nucleic Acids Res. 21:1155-62, 1993). Amine residues may be introduced onto a surface through the use of aminosilane for cross-linking.

Immobilization may take place by direct covalent attachment of 5′-phosphorylated nucleic acids or capture probes to chemically modified surfaces (Rasmussen et al., Anal. Biochem. 198:138-142, 1991). The covalent bond between the nucleic acid or probe and the surface is formed by condensation with a water-soluble carbodiimide. This method facilitates a predominantly 5′-attachment via 5′-phosphate groups.

DNA is commonly bound to glass by first silanizing the glass surface, then activating with carbodiimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule. DNA may be bound directly to membrane surfaces using ultraviolet radiation. Other non-limiting examples of immobilization techniques for nucleic acids are disclosed in U.S. Pat. Nos. 5,610,287, 5,776,674 and 6,225,068.

The type of surface to be used for immobilization of the nucleic acid is not limiting. In various embodiments, the immobilization surface may be magnetic beads, non-magnetic beads, a planar surface, or any other conformation of solid surface comprising almost any material, so long as the material is sufficiently durable and inert to allow the BRC process to occur. Non-limiting examples of surfaces that may be used include glass, silica, silicate, PDMS, silver or other metal coated surfaces, nitrocellulose, nylon, activated quartz, activated glass, polyvinylidene difluoride (PVDF), polystyrene, polyacrylamide, other polymers such as poly(vinyl chloride), poly(methyl methacrylate) or poly(dimethyl siloxane), and photopolymers which contain photoreactive species such as nitrenes, carbenes and ketyl radicals capable of forming covalent links with nucleic acids (See U.S. Pat. Nos. 5,405,766 and 5,986,076).

Bifunctional cross-linking reagents may be of use in various embodiments, such as attaching a target nucleic acid or probe to a surface. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

The skilled artisan will realize that the general cross-linking methods discussed herein are not limited to nucleic acids or oligonucleotides and may be applied, for example, to attach antibodies, binding proteins, ligands, inhibitors, substrates and/or any other compound that could be used to capture, for example, a protein-linked target oligonucleotide to a solid surface.

Fluorescent Probes and Other Labels

In certain embodiments of the invention, various labeled nucleotides may be incorporated by terminal transferase activity. Nucleotides tagged with various label moieties, such as fluorescent labels, are known in the art and may be obtained from commercial sources (e.g., Molecular Probes, Eugene, Oreg.). Alternatively, fluorophores may be conjugated to nucleotides before use. Methods for attaching fluorescent or other labels to oligonucleotide and/or DNA molecules are known in the art and any such known method may be used to make labeled probes within the scope of the present invention.

Labels of use may comprise any composition detectable by electrical, optical, spectrophotometric, photochemical, biochemical, or chemical techniques. Labels may include, but are not limited to, conducting, luminescent, fluorescent, chemiluminescent, bioluminescent and phosphorescent labels, chromogens, enzymes or substrates. Fluorescent molecules suitable for use as labels include, but are not limited to, dansyl chloride, rhodamineisothiocyanate, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red. A variety of other known fluorescent or luminescent labels may be utilized. (See, e.g., U.S. Pat. No. 5,800,992; U.S. Pat. No. 6,319,668.)

Detectors

In various embodiments of the invention, photons generated by BRC may be quantified using a detector, such as a charge coupled device (CCD). Other exemplary detectors include photodiodes, avalanche photodiodes, photomultiplier tubes, multianode photomultiplier tubes, phototransistors, vacuum photodiodes, silicon photodiodes, and CCD cameras.

In certain embodiments of the invention, a highly sensitive cooled CCD detector may be used. The cooled CCD detector has a probability of single-photon detection of up to 80%, a high spatial resolution pixel size (5 microns), and sensitivity in the visible through near infrared spectra. (Sheppard, Confocal Microscopy: Basic Principles and System Performance in: Multidimensional Microscopy, P. C. Cheng et al. eds., Springer-Verlag, New York, N.Y. pp. 1-51, 1994.) In another embodiment of the invention, a coiled image-intensified coupling device (ICCD) may be used as a photodetector that approaches single-photon counting levels (U.S. Pat. No. 6,147,198). A small number of photons triggers an avalanche of electrons that impinge on a phosphor screen, producing an illuminated image. This phosphor image is sensed by a CCD chip region attached to an amplifier through a fiber optic coupler.

In some embodiments of the invention, an avalanche photodiode (APD) may be made to detect low light levels. The APD process uses photodiode arrays for electron multiplication effects (U.S. Pat. No. 6,197,503). The invention is not limited to the disclosed embodiments and it is contemplated that any light detector known in the art that is capable of accumulating photons over a time interval may be used in the disclosed methods and apparatus.

In all of the above embodiments the generated photons from the sample can either reach the detector directly or be guided and/or focused onto the detector by a secondary system such as a number of lenses, reflecting mirror systems, optical waveguides and optical fibers or a combination of those.

### EXAMPLES

### Example 1

### BRC Assay With Terminal Transferase

Sample Preparation

In an exemplary embodiment, a reporter oligonucleotide (e.g., d(A)₁₈) may be covalently attached to a secondary antibody, for example a goat anti-mouse antibody, using known techniques (e.g., Schweitzer et al. Proc. Natl. Acad. Sci. USA 97:10113-119, 2000). Target proteins to be detected in a sample may be immobilized on a substrate using standard methods, as discussed above. A mouse monoclonal antibody specific for a given target protein may be added and allowed to bind to the target. After washing, the oligonucleotide-tagged goat anti-mouse antibody may be added and allowed to bind to the mouse monoclonal antibody attached to the target protein. Excess secondary antibody may be removed by washing. Many variations on this scheme, such as sandwich ELISA, are known in the art and may be utilized.

Terminal transferase (0.1 mU) may be added to the bound reporter oligonucleotide in buffer (20 mM Tris acetate, pH 7.9, 50 mM potassium acetate, 1.5 mM COCl₂) with 0.2 mM alpha-thio dATP in a 50 μl reaction volume. The terminal transferase reaction may be terminated by heating at 70° C. for 15 min and then chilling on ice.

An aliquot containing PPi may be added to 50 μl of reaction mixture (see Ronaghi et al., Anal. Biochem. 242:84-89, 1996 with modifications) containing 250 ng luciferase (Promega, Madison, Wis.), 50 mU ATP sulfurylase (Sigma Chemical Co., St. Louis, Mo.), 2 mM dithiothreitol, 100 mM Tris-Acetate pH 7.75, 0.5 mM EDTA, 0.5 mg BSA, 0.2 mg polyvinylpyrrolidone (Mᵣ 360,000), 10 μg D-luciferin (Biothema, Dalaro, Sweden), 5 mM magnesium acetate and 10 attomole to 0.01 attomole purified pyrophosphate or ATP. The addition of very low amounts of pyrophosphate or ATP (or analogs) is important to decrease background light emission from the reaction mixture. Although the precise mechanism is unknown, BRC performed without adding small amounts of ATP or PPi exhibits background luminescence that precludes accurate measurement of target molecules present in amounts of about a femtomole or lower. Inorganic pyrophosphate present in the sample as a result of terminal transferase mediated dNTP incorporation may be converted to ATP by sulfurylase. The ATP may be used to generate light in a luciferin/luciferase reaction.

Detection Devices

The number of the photons generated by BRC may be measured using any known type of photodetector. Common devices that may be used include photodiodes, photomultiplier tubes (PMTs), charge coupled devices (CCDs), and photo-resistive materials. Luciferase-catalyzed photon generation has a quantum yield (Q.E.) of approximately 0.88, with the wavelength maximum depending on the type of luciferase used. For various types of luciferase, that can be anyplace within the visible range of the spectrum. Exemplary embodiments use firefly luciferase, which has a maximum intensity at 562 nm.

The photosensitive device is typically either in direct proximity of the BRC reaction to directly receive incident photons, or relatively far from the buffer with a light coupling device (e.g. optical fiber or mirror system) capable of directing light from the sample to the detector. In an exemplary embodiment, a UDT-PIN-UV-50-9850-1 photodiode (Hamamatsu Corp., Hamamatsu, Japan) may be used with a transimpedance amplifier with a gain of 10⁸ volts/amp.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method comprising:
a) obtaining at least one sample suspected of containing one or more target nucleic acids;
b) generating pyrophosphate (PPi) by terminal transferase;
c) producing light by a bioluminescence regenerative cycle (BRC); and
e) detecting the target nucleic acid.

2. The method of claim 1, further comprising identifying the target nucleic acid.

3. The method of claim 1, further comprising determining the amount of target nucleic acid in the sample.

4. The method of claim 1, wherein the target nucleic acid comprises genomic DNA.

5. The method of claim 1, wherein the target nucleic acid comprises cDNA.

6. The method of claim 1, wherein the target nucleic acid comprises a single nucleotide polymorphism (SNP) site.

7. The method of claim 1, further comprising immobilizing the target nucleic acid with a sequence specific oligonucleotide capture probe.

8. The method of claim 1, wherein the target nucleic acid comprises a reporter oligonucleotide attached to a biomolecule.

9. The method of claim 8, wherein the biomolecule is a protein, peptide, antibody, antibody fragment, aptamer, enzyme, inhibitor, substrate, antigen or ligand.

10. The method of claim 9, further comprising immobilizing the biomolecule on a surface.

11. The method of claim 10, wherein the biomolecule is immobilized by attachment to an antibody.

12. The method of claim 1, further comprising measuring gene expression levels in a sample from a cell line, tissue, organ or subject.

13. The method of claim 11, further comprising measuring the expression of two or more genes.

14. The method of claim 1, further comprising detecting a pathogen DNA sequence.

15. The method of claim 14, wherein the pathogen is selected from Table 1.

16. The method of claim 1, further comprising isolating messenger RNA (mRNA) from a sample.

17. The method of claim 15, further comprising converting the mRNA into complementary DNA (cDNA).

18. The method of claim 1, wherein the bioluminescence regenerative cycle utilizes adenosine 5′-phosphosulphate (APS), ATP sulfurylase, luciferin and luciferase.

19. The method of claim 18, further comprising adding ATP or PPi to the sample before light is produced.

20. The method of claim 1, further comprising determining the amount of target nucleic acid in the sample by integration of photon emission over a time interval.

21. The method of claim 1, wherein the terminal transferase reaction is terminated before the BRC assay.

22. The method of claim 1, wherein the terminal transferase reaction occurs simultaneously with the BRC assay.

23. The method of claim 1, wherein the terminal transferase is a thermostable terminal transferase.

24. A method for biomolecule detection comprising:
a) generating pyrophosphate in a biomolecule dependent process;
b) using thermostable ATP sulfurylase and luciferase to produce light from the pyrophosphate; and
c) measuring the light output to detect the biomolecule.

25. The method of claim 24, wherein the biomolecule is an oligonucleotide, polynucleotide or nucleic acid.

26. The method of claim 25, wherein the biomolecule dependent process comprises DNA polymerase activity, polymerase chain reaction amplification (PCR™), real time PCR, reverse transcriptase activity or terminal transferase activity.

27. The method of claim 24, wherein the biomolecule is a protein, peptide, antibody, antibody fragment, enzyme, receptor protein, ligand, substrate or inhibitor.

28. The method of claim 27, wherein the biomolecule is attached to an oligonucleotide.

29. A method comprising:
a) obtaining at least one sample suspected of containing one or more target nucleic acids;
b) adding labeled nucleotides to the one or more target nucleic acids with a thermostable terminal transferase; and
c) detecting the labeled nucleic acids.

30. The method of claim 29, wherein each type of nucleotide is labeled with a distinguishable label.

31. The method of claim 29, wherein the nucleotides are labeled with one or more fluorophores.

32. The method of claim 31, wherein different types of nucleotides are labeled with fluorophores of different color.

33. A method of biomolecule detection comprising:
a) attaching a target molecule to a substrate;
b) binding a first binding moiety to the target molecule;
c) binding a second binding moiety to the first binding moiety, wherein the second binding moiety is attached to dextran labeled with oligonucleotides;
d) generating pyrophosphate by terminal transferase mediated addition of nucleotides to the oligonucleotides; and
e) detecting the pyrophosphate.

34. The method of claim 33, wherein the pyrophosphate is detected by BRC assay.

35. The method of claim 33, wherein the terminal transferase is a thermostable terminal transferase.
